(19) United States
(12) Patent Application Publication
(10) Pub. No.: US 2023/0059769 A1
(43) Pub. Date: Feb. 23, 2023

(54) FACIAL SKIN DISORDER IDENTIFICATION SYSTEM

(71) Applicants: BUDDHIST TZU CHI MEDICAL FOUNDATION, Haulien (TW); NATIONAL APPLIED RESEARCH LABORATORIES, Taipei City (TW)

(72) Inventors: Chung-Hsing Chang, Hualien (TW); Shinn-Zong Lin, Haulien (TW); Chun-Ming Chang, Taipei City (TW); Nai-Yuan Chiang, Taipei City (TW); Rou-Jhen Chen, Taipei City (TW); Han-Chao Chang, Taipei City (TW)

(73) Assignees: BUDDHIST TZU CHI MEDICAL FOUNDATION, Haulien (TW); NATIONAL APPLIED RESEARCH LABORATORIES, Taipei City (TW)

(21) Appl. No.: 17/649,069

(22) Filed: Jan. 27, 2022

(30) Foreign Application Priority Data
Aug. 23, 2021 (TW) 110131086

(51) Int. Cl. G06T 7/00; H04N 5/225; H04N 5/232; G06T 11/00; G06T 7/73; G03B 17/56; H01F 7/06; G01D 5/347; A61B 5/107; A61B 5/00; A61N 5/06; G16H 30/20; G16H 50/50

(52) U.S. Cl. CPC G06T 7/0012; H04N 5/2256; H04N 5/23229; G06T 11/00; G06T 7/75; H04N 5/2253; H04N 5/23299; H04N 5/23222; G03B 17/561; H01F 7/064; G01D 5/34746; A61B 5/1077; A61B 5/0077; A61B 5/6844; A61B 5/441; A61N 5/0616; A61N 5/062; A61B 5/4836; A61B 5/6814; G16H 30/20; G16H 50/50; G06T 2207/30201; G06T 2207/30088; A61N 2005/0647; A61N 2005/0663

(57) ABSTRACT

A facial skin disorder (FSD) identification system is provided and includes a sliding rail arranged around a human facial epidermis, a carrier arranged on the sliding rail, at least one image capturing device arranged on the carrier, and a control circuit unit arranged on the carrier for the carrier to move on the sliding rail and the image capturing device to capture images of the human facial epidermis.

### BACKGROUND

### 1. Technical Field

The present disclosure relates to a facial skin disorder (FSD) identification system, and more particularly, to a facial skin disorder identification system for actinic keratosis.

### 2. Description of Related Art

Actinic keratosis is a skin disorder that tends to occur in middle-aged or older adults. The lesions on the skin develop from years of sun exposure. For instance, the skin surface of the affected area is rough or peeling and appears scaly patch or raised bumps, and these bumps may be mostly red, brown or the same color as your skin. Therefore, actinic keratosis is often misdiagnosed as age spots or eczema. In addition to simply examining the skin with a simple observation method, a dermatologist may also use an auxiliary medical device such as a dermatoscope to assist in the diagnosis of actinic keratosis.

However, the conventional dermoscopy still has several problems. For example, light is easily scattered when using the conventional dermoscopy during a close-up examination of the skin of the patient. Further, the images observed by the dermatologist cannot be stored as files in a computer for the patient's next appointment. Therefore, there is an urgent need to provide a facial skin disorder identification system that solves the abovementioned problems of the prior art.

### SUMMARY

In view of the aforementioned problems of the prior art, the present disclosure provides a facial skin disorder identification system, which comprises: a sliding rail arranged around a human facial epidermis; a carrier arranged on the sliding rail; at least one image capturing device arranged on the carrier; and a control circuit unit arranged on the carrier for the carrier to move on the sliding rail and the image capturing device to capture images of the human facial epidermis.

In the aforementioned facial skin disorder identification system, the sliding rail has at least one sensing unit sequentially arranged on a surface of the sliding rail, wherein the control circuit unit faces the sensing unit and is arranged on the carrier, and wherein the control circuit unit is configured for the carrier to slide on the sliding rail according to the sensing unit.

In the aforementioned facial skin disorder identification system, the plurality of sensing units are electromagnets, wherein the control circuit unit controls generation of magnetism of the plurality of sensing units by charging and discharging for the carrier to slide on the sliding rail.

In the aforementioned facial skin disorder identification system, the carrier is a U-shaped structure having two opposite side walls, wherein a first groove is respectively arranged on opposite sides of the two side walls.

In the aforementioned facial skin disorder identification system, a second groove corresponding to the first groove is respectively provided on two opposite surfaces of the sliding rail, wherein after the carrier is arranged on the sliding rail, the first groove and the second groove form a cylindrical groove together and accommodate at least two balls, respectively.

In the aforementioned facial skin disorder identification system, the sensing unit is an optical encoder, and the control circuit unit is an optical read head including a control circuit and a stepping motor, wherein after the optical read head generates a signal of each position on the optical encoder, the control circuit unit controls the stepping motor according to the signal for the carrier to slide on the sliding rail.

In the aforementioned facial skin disorder identification system, the present disclosure further comprises a computer device electrically connected to the image capturing device and the control circuit unit, the computer device comprising: a storage module configured to store the images captured by the image capturing device; a position recording module configured to record position information of the image capturing device, wherein the position information includes a position of the image capturing device when the image capturing device captures the images of the human facial epidermis and serial numbers of the images captured at the position; a modeling module configured to model according to the images of the human facial epidermis captured by the image capturing device to form a human facial epidermis model; and a marking module configured to perform feature point marking on the human facial epidermis model.

In the aforementioned facial skin disorder identification system, the human facial epidermis model is a three-dimensional model or a two-dimensional plane image.

In the aforementioned facial skin disorder identification system, the present disclosure further comprises a linear sliding bar configured to connect the sliding rail for the sliding rail to move or rotate on the linear sliding bar.

In the aforementioned facial skin disorder identification system, the computer device further comprises a detailed photographing module configured to obtain the position information according to the images corresponding to the feature point marking, wherein the sliding rail is moved or rotated on the linear sliding bar to shorten a distance between the sliding rail and the human facial epidermis, and wherein after the image capturing device is moved to the position corresponding to the sliding rail in the position information, the image capturing device performs detailed photographing to the human facial epidermis.

In the aforementioned facial skin disorder identification system, when the control circuit unit enables the image capturing device to capture the images of the human facial epidermis or to perform detailed photographing of the human facial epidermis an image capturing light source of the image capturing device is an ordinary white light or a natural light, wherein when the detailed photographing module enables the image capturing device to perform photodynamic diagnostic photographing of the human facial epidermis, the image capturing light source of the image capturing device is a light source in a wavelength range from 365 nm to 410 nm, and wherein when the detailed photographing module performs a photodynamic therapy on a position of the feature point marking after photographing is completed, a photodynamic therapy light source in a wavelength range from 600 nm to 700 nm is used for the photodynamic therapy.

In the aforementioned facial skin disorder identification system, the image capturing device is a visible light camera, a structured light camera, or a macro camera.

In the aforementioned facial skin disorder identification system, the sliding rail is a curved-line sliding rail or a curved-face sliding rail that conforms to a three-dimensional curvature of the human facial epidermis.

In the aforementioned facial skin disorder identification system, the sliding rail is defined with a plurality of coordinate codes, wherein the carrier includes an encoder motor, and wherein the control circuit unit is configured for the carrier to move sequentially on the plurality of coordinate codes of the sliding rail and is configured for the image capturing device to sequentially capture the images of the human facial epidermis on the plurality of coordinate codes according to an image capturing frequency of the image capturing device and a rotation speed of the encoder motor.

In the aforementioned facial skin disorder identification system, one side of the sliding rail is arranged with a flexible arc-shaped rack including a plurality of tooth pitches, wherein the carrier includes a gear that matches the plurality of tooth pitches, wherein the control circuit unit is configured to rotate the gear according to an image capturing frequency of the image capturing device and the plurality of tooth pitches for the carrier to move sequentially on the plurality of tooth pitches and for the image capturing device to sequentially capture the images of the human facial epidermis on the plurality of tooth pitches.

### BRIEF DESCRIPTION OF THE DRAWINGS

FIG. 1 is a schematic view of a facial skin disorder identification system according to an embodiment of the present disclosure.

FIG. 2 is a schematic view of a facial skin disorder identification system according to another embodiment of the present disclosure.

FIG. 3 is a schematic view of a human facial epidermis model in a facial skin disorder identification system of the present disclosure.

FIG. 4 is a schematic view of a facial skin disorder identification system performing detailed photographing of the present disclosure.

FIG. 5 is a schematic overall structure view of a sliding rail and a carrier of a facial skin disorder identification system of the present disclosure.

FIG. 6 is a schematic cross-sectional view of the sliding rail and the carrier along the A-A section line of FIG. 5.

FIG. 7 is a system architecture view of a facial skin disorder identification system including a computer device of the present disclosure.

### DETAILED DESCRIPTION OF THE EMBODIMENTS

The following examples describe the embodiments of the present disclosure. A skilled person in the art can understand advantages and effects of the present disclosure from the content disclosed in the specification and can also be implemented or applied by other different embodiments.

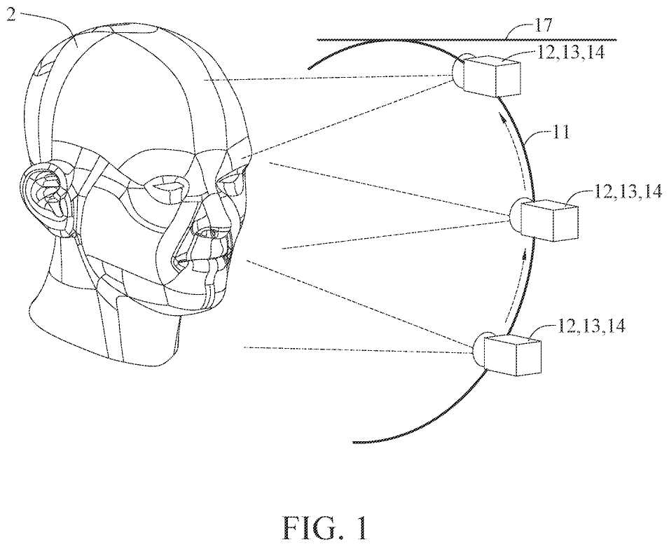

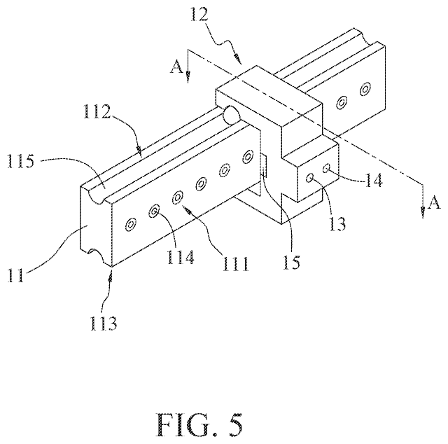

Please refer to FIG. 1 and FIG. 5 at the same time, where FIG. 1 is a schematic view of a facial skin disorder (FSD) identification system according to an embodiment of the present disclosure, and FIG. 5 is a schematic overall structure view of a sliding rail and a carrier in the facial skin disorder identification system according to the present disclosure. The facial skin disorder identification system according to the present disclosure includes a sliding rail 11, a carrier 12, at least one image capturing device 13 and 14, and a control circuit unit 15. The structure of the sliding rail 11, the carrier 12 and the image capturing devices 13 and 14 can be understood by referring to FIG. 5. The sliding rail 11, the carrier 12 and the image capturing devices 13 and 14 depicted in FIG. 1 are examples intended to illustrate that the carrier 12 and the image capturing devices 13 and 14 may move on the sliding rail 11 to perform image capturing operations from parts of a human facial epidermis 2 at different positions and angles.

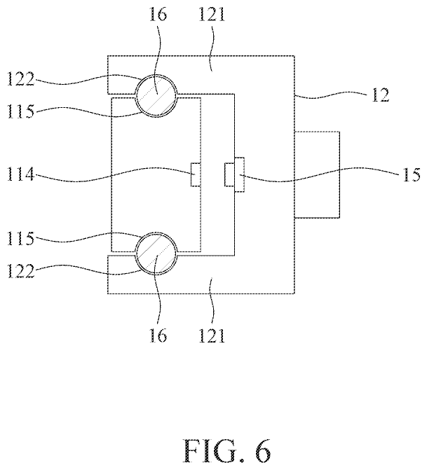

In an embodiment, the sliding rail 11 is arranged around the human facial epidermis 2, and the carrier 12 is arranged on the sliding rail 11. Further, the image capturing devices 13 and 14 may be arranged on the carrier 12. As shown in FIG. 5 and FIG. 6, the sliding rail 11 has a plurality of sensing units 114 sequentially arranged on a surface 111 thereof, and a second groove 115 corresponding to a first groove 122 is respectively provided on two opposite surfaces 112, 113 adjacent to the surface 111. In an embodiment, the second groove 115 is a semi-cylindrical groove recessed in the surfaces 112, 113.

As shown in FIG. 5 and FIG. 6, the carrier 12 is a U-shaped structure having two opposite side walls 121, and the first groove 122 is respectively arranged on opposite sides of the two side walls 121. In an embodiment, the first groove 122 may be a semi-cylindrical groove recessed in the side wall 121. Furthermore, after the carrier 12 is arranged on the sliding rail 11, the first groove 122 and the second groove 115 can correspondingly form a cylindrical groove together, and can respectively accommodate at least two balls 16. As such, the carrier 12 may move on the sliding rail 11 via the balls 16, the first groove 122 and the second groove 115.

In an embodiment, the control circuit unit 15 may be arranged on the carrier 12. For instance, the control circuit unit 15 may be arranged on a surface of the carrier 12 between the two side walls 121 and faces the plurality of sensing units 114. In an embodiment, the plurality of sensing units 114 may be electromagnets, and the control circuit unit 15 may be a programmed circuit board (PCB). The control circuit unit 15 may provide power to the plurality of sensing units 114 and control the generation of magnetism of the plurality of sensing units 114 by charging and discharging so as to enable the carrier 12 to move on the sliding rail 11, so that the carrier 12 may be moved arbitrarily to the desired position on the sliding rail 11 and the image capturing devices 13 and 14 may capture images of the human facial epidermis 2.

For example, the control circuit unit 15 may be supplied with positive electricity, and the sensing units 114 may be supplied with negative electricity. The carrier 12 may move on the sliding rail 11 when the power is on and may stop at a position of the sliding rail 11 when the power is off; and at this time, the image capturing devices 13 and 14 may capture images of the human facial epidermis 2 to obtain a plurality of images.

In the abovementioned embodiment, the sensing units 114 are electromagnets that allow the carrier 12 to move on the sliding rail 11 by an electromagnetic moving method. However, the present disclosure may also use a moving method such as an encoder motor, an optical encoder (e.g., programmable), or a gear arc-shaped rack system, and the present disclosure is not limited thereto. The following describes various embodiments of different moving methods.

In other embodiments, the sensing unit 114 may be an optical encoder, which may be attached on the surface 111 of the sliding rail 11. The control circuit unit 15 may be an optical read head that includes a control circuit and a stepping motor. A plurality of parallel lines with equal intervals may be engraved on the optical encoder. The optical read head may be configured to read the parallel lines on the optical encoder so as to generate a signal of each position on the optical encoder. The control circuit unit may control the stepping motor according to the signal so as to enable the carrier 12 to slide on the sliding rail 11. Moreover, the control circuit unit may also enable the image capturing devices 13 and 14 to sequentially capture images of the human facial epidermis 2 on each parallel line according to the plurality of parallel lines with equal intervals, the rotation speed of the stepping motor, and the image capturing frequency of the image capturing devices 13 and 14, thereby obtaining a plurality of images.

In another embodiment, the sensing units 114 may not be required to use. Instead, an encoder motor may be provided in the carrier 12 and configured to encode the path coordinates (or encode X-Y coordinates) of the total length of the sliding rail 11. Therefore, the sliding rail 11 may be defined with a plurality of coordinate codes, so that the position of each point or coordinate on the sliding rail 11 may be known, and the control circuit unit 15 may control the movement of the carrier 12 to each point position via the encoder motor. Furthermore, the control circuit unit 15 may be configured to enable the carrier 12 to move sequentially on each coordinate code of the sliding rail 11 according to the image capturing frequency of the image capturing devices 13 and 14 and the rotation speed of the encoder motor, and simultaneously make the image capturing devices 13 and 14 to sequentially capture images of the human facial epidermis 2 on each coordinate code, thereby obtaining a plurality of images.

In another embodiment, the sensing units 114 may not be required to use, and a flexible arc-shaped rack is provided on one side of the sliding rail 11, where the flexible arc-shaped rack includes a plurality of tooth pitches. The carrier 12 may include a gear that matches the plurality of tooth pitches, so that the carrier 12 may move on the sliding rail 11 synchronously when the gear rotates, and the position of each point of the sliding rail 11 may be defined by the plurality of tooth pitches (e.g., calculating the moving distance of the carrier 12 via the number of tooth pitches, etc. to define each point position). In addition, the control circuit unit 15 is configured to rotate the gear according to the image capturing frequency of the image capturing devices 13 and 14 and the plurality of tooth pitches, so as to enable the carrier 12 to move sequentially on the plurality of tooth pitches, and simultaneously make the image capturing devices 13 and 14 to sequentially capture images of the human facial epidermis 2 on the plurality of tooth pitches, thereby obtaining a plurality of images.

In an embodiment, the image capturing devices 13 and 14 may be visible light cameras, structured light cameras, or macro cameras, and the present disclosure is not limited thereto.

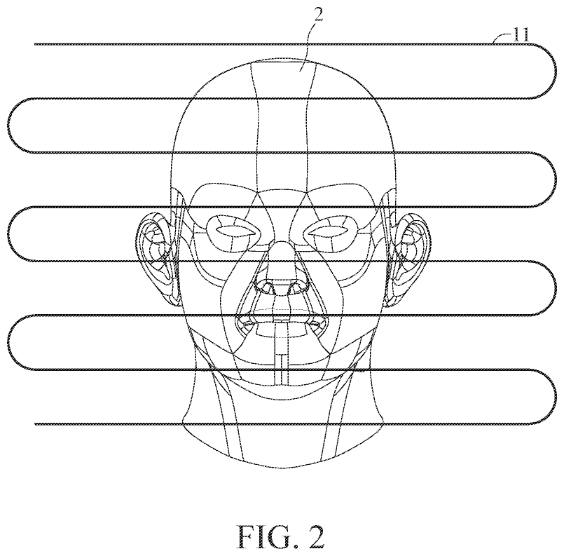

In an embodiment, the sliding rail 11 may be a curved-line sliding rail that conforms to the three-dimensional curvature of the human facial epidermis 2. In another embodiment, as shown in FIG. 2, the sliding rail 11 may be a curved-face sliding rail, which may be arranged in front of the human facial epidermis 2 in multiple bending sections, so that the image capturing devices 13 and 14 on the carrier 12 may capture images of the human facial epidermis 2 from multiple angles and positions, but the present disclosure is not limited thereto.

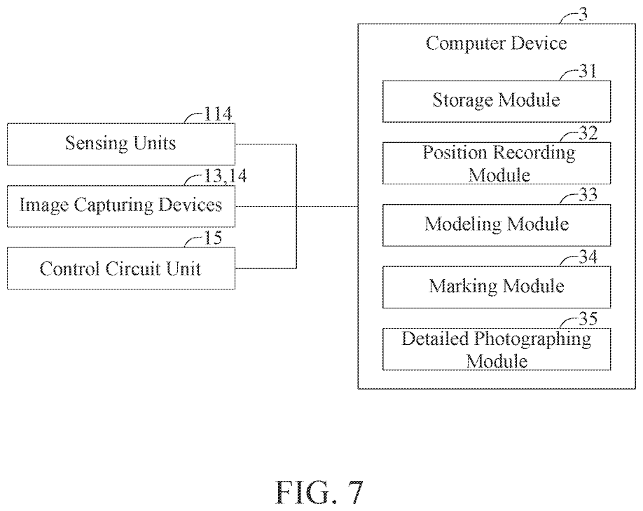

Referring to FIG. 7, the facial skin disorder identification system according to the present disclosure may further include a computer device 3. The computer device 3 may be electrically connected to the image capturing devices 13 and 14 and the control circuit unit 15, for example, through a wireless network (Wi-Fi, Bluetooth, 3G, 4G, 5G, etc.) or wired network connection. The computer device 3 may include a storage module 31, a position recording module 32, a modeling module 33, a marking module 34, and a detailed photographing module 35. In an embodiment, the storage module 31 may be a hardware such as a hard disk, a floppy disk, or a memory, etc., and the position recording module 32, the modeling module 33, the marking module 34 and the detailed photographing module 35 may be software run by a microprocessor. Further, if the computer device 3 is in the embodiment having the sensing units 114, the computer device 3 may also be electrically connected with the plurality of sensing units 114.

In an embodiment, the storage module 31 is configured to store the images captured by the image capturing devices 13 and 14. The position recording module 32 may be configured to record the position information of the image capturing devices 13 and 14, where the position information may include a position of the image capturing devices 13 and 14 when the image capturing devices 13 and 14 capture the images of the human facial epidermis 2 and the serial numbers of the images captured at the position. For example, when the sensing units 114 are optical encoders, the signal generated by different parallel lines may be used to confirm the current position of the carrier 12 on the sliding rail 11, and the serial numbers (e.g., file names) of the images captured at the position may be linked with the corresponding signal. As such, the corresponding images may be found via the read signal of the corresponding parallel lines of the carrier 12 on the sliding rail 11, and the images may also be used to move the carrier 12 to the position of the sliding rail 11 when the images are captured. For another example, when the sensing units 114 are not used, each point position of the sliding rail 11 may be calculated via the plurality of coordinate codes defined by the encoder motor or the flexible arc-shaped rack including the plurality of tooth pitches, and the serial number of different images captured at each point position may be linked with each corresponding point position. As such, images may be used to move the carrier 12 to the position of the sliding rail 11 when the images are captured. For yet another example, when the sensing units 114 are electromagnets, the control circuit unit 15 has a timing chip (e.g., a real-time clock) therein. When the carrier 12 moves, the control circuit unit 15 may record the time and match the image capturing time to complete the image capturing operation of the human facial epidermis 2. The time point of the image captured is the position of the image captured (usually the same as the position of the sensing units 114).

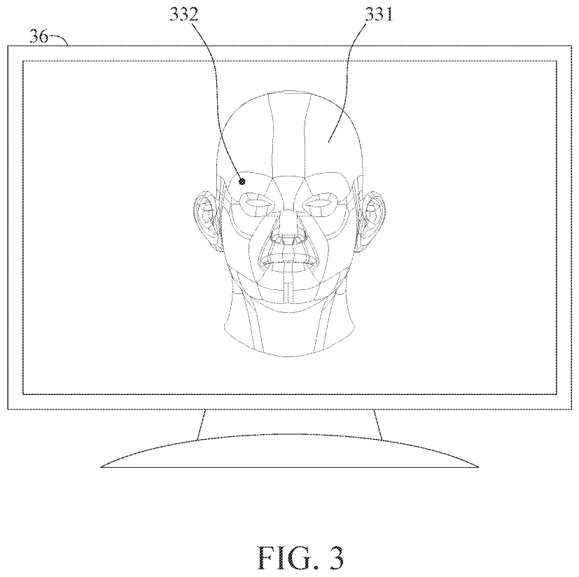

In an embodiment, the modeling module 33 is configured to model according to a plurality of images of the human facial epidermis 2 captured by the image capturing devices 13 and 14 on each point position of the sliding rail 11 so as to form a human facial epidermis model, e.g., a human facial epidermis model 331 presented in a screen 36 as shown in FIG. 3. In an embodiment, the human facial epidermis model 331 modeled by the modeling module 33 may be a three-dimensional (3D) model; for example, it may be modeled by using a general 3D modeling software available on the market. At this time, one of the image capturing devices 13 and 14 may be configured as a structured light camera to obtain the depth information of the human facial epidermis 2 required by the 3D modeling software. In another embodiment, the human facial epidermis model 331 modeled by the modeling module 33 may be a two-dimensional (2D) plane image (e.g., a general imaging software on the market may be used to merge a plurality of images into one image). At this time, one of the image capturing devices 13 and 14 may be configured as a visible light camera.

As shown in FIG. 3, after the modeling module 33 generates the human facial epidermis model 331, the human facial epidermis model 331 may be presented on the screen 36. A user may perform a feature point marking 332 on the human facial epidermis model 331 via the marking module 34 and use the detailed photographing module 35 (e.g., shown in FIG. 7) to drive the carrier 12 to the human facial epidermis position corresponding to the feature point marking 332 for detailed photographing in order to conduct further observation and comparison. For example, the detailed photographing module 35 may obtain the aforementioned position information according to the images corresponding to the feature point marking 332. As such, the image capturing devices 13 and 14 may slide to the position corresponding to the sliding rail 11 in the position information and perform detailed photographing to the human facial epidermis 2. At this time, one of the image capturing devices 13 and 14 may be a macro camera. In other words, the combination of the image capturing devices 13 and 14 may be a visible light camera and a macro camera, or a structured light camera and a macro camera, but the present disclosure is not limited thereto.

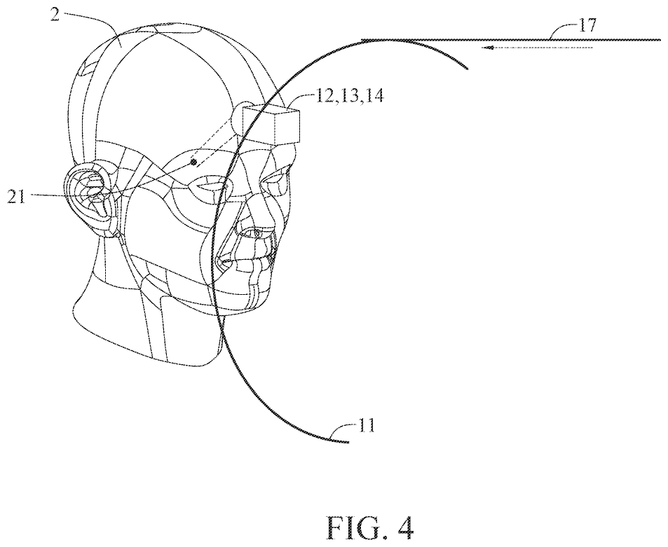

In another embodiment, in order to allow the image capturing devices 13 and 14 to be closer to the human facial epidermis 2 for detailed photographing, the facial skin disorder identification system of the present disclosure further includes a linear sliding bar 17 for connecting the sliding rail 11, so that the sliding rail 11 may slide or rotate on the linear sliding bar 17 (e.g., the sliding range of the sliding rail 11 is between ±80 degrees). As shown in FIG. 4, when performing detailed photographing, after the detailed photographing module 35 obtains the aforementioned position information according to the images corresponding to the feature point marking 332, the sliding rail 11 may first be moved or rotated on the linear sliding bar 17 to minimize the distance between the sliding rail 11 and the human facial epidermis 2, and then allow the movement of the image capturing devices 13 and 14 and proceed with detailed photographing. The relative moving or rotating method of the linear sliding bar 17 on the sliding rail 11 described herein may be any of the aforementioned electromagnetic, encoder motor, optical encoder, or gear arc-shaped rack system moving method.

In an embodiment, the light source used in the facial skin disorder identification system according to the present disclosure is that: (1) the image capturing light source of the image capturing devices 13 and 14 may be ordinary white light or natural light, which is the main image capturing light source for actinic keratosis; (2) the photodynamic diagnostic light source is a light source in the wavelength range from 365 nm to 410 nm. After obtaining the images of the suspected skin diseases (such as actinic keratosis, which is identified by a self-developed AI program on human facial epidermis 2) and applying photodynamic drugs, the computer device 3 may control the image capturing devices 13 and 14 to the position of the corresponding feature point marking 332 to capture the images with a light source in the wavelength range from 365 nm to 410 nm (i.e., the detailed photographing operation of the abovementioned detailed photographing module 35, and for example, a yellow filter may be arranged on the image capturing devices 13 and 14 before capturing images); (3) after taking images with a light source in the wavelength range from 365 nm to 410 nm, the physician may determine which position of the feature point marking 332 has a drug response, and use the photodynamic therapy light source in the wavelength range from 600 nm to 700 nm to perform photodynamic therapy.

In summary, the facial skin disorder identification system of the present disclosure is based on a sliding rail design to drive the image capturing devices on the carrier to create a human facial epidermis model of a patient, and provides the user (e.g., dermatologist) to perform feature point marking on the human facial epidermis model so as to take detailed photographs of the feature point marking. The enlarged image obtained by the detailed photographing may assist the user to compare the affected area of the patient, so that the user may make more accurate diagnosis and treatment opinions.

The above-described descriptions of the detailed embodiments are to illustrate the implementation according to the present disclosure, and it is not to limit the scope of the present disclosure. Accordingly, all modifications and variations completed by those with ordinary skill in the art should fall within the scope of the present disclosure defined by the appended claims.

What is claimed is:

1. A facial skin disorder identification system, comprising:
a sliding rail configured to be arranged around a human facial epidermis;
a carrier arranged on the sliding rail;
at least one image capturing device arranged on the carrier; and
a control circuit unit arranged on the carrier for the carrier to move on the sliding rail and the image capturing device to capture images of the human facial epidermis.

2. The facial skin disorder identification system of claim 1, wherein the sliding rail has at least one sensing unit sequentially arranged on a surface of the sliding rail, wherein the control circuit unit faces the sensing unit and is arranged on the carrier, and wherein the control circuit unit is configured for the carrier to slide on the sliding rail according to the sensing unit.

3. The facial skin disorder identification system of claim 2, wherein the sensing unit is an electromagnet, and the control circuit unit is configured to control generation of magnetism of the sensing unit by charging and discharging for the carrier to slide on the sliding rail.

4. The facial skin disorder identification system of claim 3, wherein the carrier is a U-shaped structure having two opposite side walls, and wherein a first groove is respectively arranged on opposite sides of the two side walls.

5. The facial skin disorder identification system of claim 4, wherein a second groove corresponding to the first groove is respectively provided on two opposite surfaces of the sliding rail, and wherein after the carrier is arranged on the sliding rail, the first groove and the second groove form a cylindrical groove and respectively accommodate at least two balls.

6. The facial skin disorder identification system of claim 2, wherein the sensing unit is an optical encoder, and the control circuit unit is an optical read head including a control circuit and a stepping motor, and wherein after the optical read head generates a signal of each position on the optical encoder, the control circuit controls the stepping motor according to the signal for the carrier to slide on the sliding rail.

7. The facial skin disorder identification system of claim 1, further comprising a computer device electrically connected to the image capturing device and the control circuit unit, the computer device comprising:
a storage module configured to store the images captured by the image capturing device;
a position recording module configured to record position information of the image capturing device, wherein the position information includes a position of the image capturing device when the image capturing device captures the images of the human facial epidermis and serial numbers of the images captured at the position;
a modeling module configured to model according to the images of the human facial epidermis captured by the image capturing device to form a human facial epidermis model; and
a marking module configured to perform feature point marking on the human facial epidermis model.

8. The facial skin disorder identification system of claim 7, wherein the human facial epidermis model is a three-dimensional model or a two-dimensional plane image.

9. The facial skin disorder identification system of claim 7, further comprising a linear sliding bar configured to connect the sliding rail for the sliding rail to move or rotate on the linear sliding bar.

10. The facial skin disorder identification system of claim 9, wherein the computer device further comprises a detailed photographing module configured to obtain the position information according to the images corresponding to the feature point marking, wherein the sliding rail is moved or rotated on the linear sliding bar to shorten a distance between the sliding rail and the human facial epidermis, and wherein after the image capturing device is moved to the position corresponding to the sliding rail in the position information, the image capturing device performs detailed photographing to the human facial epidermis.

11. The facial skin disorder identification system of claim 10, wherein when the control circuit unit is configured for the image capturing device to capture the images of the human facial epidermis or to perform detailed photographing of the human facial epidermis, an image capturing light source of the image capturing device is an ordinary white light or a natural light, wherein when the detailed photographing module is configured for the image capturing device to perform photodynamic diagnostic photographing of the human facial epidermis, the image capturing light source of the image capturing device is a light source in a wavelength range from 365 nm to 410 nm, and a photodynamic therapy light source in a wavelength range from 600 nm to 700 nm is used for the photodynamic therapy when the detailed photographing module is configured to perform a photodynamic therapy on a position of the feature point marking after photographing is completed.

12. The facial skin disorder identification system of claim 1, wherein the image capturing device is a visible light camera, a structured light camera, or a macro camera.

13. The facial skin disorder identification system of claim 1, wherein the sliding rail is a curved-line sliding rail or a curved-face sliding rail that conforms to a three-dimensional curvature of the human facial epidermis.

14. The facial skin disorder identification system of claim 1, wherein the sliding rail is defined with a plurality of coordinate codes, wherein the carrier includes an encoder motor, and wherein the control circuit unit is configured for the carrier to move sequentially on the plurality of coordinate codes of the sliding rail and is configured for the image capturing device to sequentially capture the images of the human facial epidermis on the plurality of coordinate codes according to an image capturing frequency of the image capturing device and a rotation speed of the encoder motor.

15. The facial skin disorder identification system of claim 1, wherein one side of the sliding rail is arranged with a flexible arc-shaped rack comprising a plurality of tooth pitches, wherein the carrier includes a gear matching the plurality of tooth pitches, wherein the control circuit unit is configured to rotate the gear according to an image capturing frequency of the image capturing device and the plurality of tooth pitches for the carrier to move sequentially on the plurality of tooth pitches and for the image capturing device to sequentially capture the images of the human facial epidermis on the plurality of tooth pitches.
